(19) United States
(12) Patent Application Publication
(10) Pub. No.: US 2012/0197133 A1
(43) Pub. Date: Aug. 2, 2012

(54) Advanced Ultrasound Modulated Optical Spectroscopy And Its Application To Patient Monitoring

(75) Inventors: Edward M. McKenna, Boulder, CO (US); Youzhi Li, Longmont, CO (US); Andy S. Lin, Boulder, CO (US)

(73) Assignee: Nellcor Puritan Bennett LLC, Boulder, CO (US)

(21) Appl. No.: 13/016,629

(22) Filed: Jan. 28, 2011

(51) Int. Cl. A61B 6/00

(52) U.S. Cl. 600/476

(57) ABSTRACT

The present disclosure describes systems and methods that use spatial modulation to focus light into a localized region of interest such as an individual blood vessel. In certain embodiments, acoustic modulation techniques, such as ultrasound pulse modulation, are used in conjunction with spatial modulation to achieve more precise measurements through otherwise scattering medium. The focused beam of light is capable of penetrating several centimeters of tissue to deliver measurements and images associated with individual blood vessels and other discrete vascular components.

### BACKGROUND

The present disclosure relates generally to optical spectroscopy and, more particularly, to ultrasound modulated optical spectroscopy.

This section is intended to introduce the reader to various aspects of art that may be related to various aspects of the present disclosure, which are described and/or claimed below. This discussion is believed to be helpful in providing the reader with background information to facilitate a better understanding of the various aspects of the present disclosure. Accordingly, it should be understood that these statements are to be read in this light, and not as admissions of prior art.

In the field of medicine, doctors often desire to monitor certain physiological characteristics of their patients. Accordingly, a wide variety of devices have been developed for monitoring many such characteristics of a patient. Such devices provide doctors and other healthcare personnel with the information they need to provide the best possible healthcare for their patients. As a result, such monitoring devices have become an indispensable part of modern medicine.

Certain monitoring devices, for example, spectroscopy devices, are capable of measuring different physiological parameters, including oxygen saturation, hemoglobin, blood perfusion, and so forth. Spectroscopy devices typically irradiate a light into a patient tissue. The irradiated region usually encompasses a wide array of blood vessels such as arterioles and capillaries. Absorbance data at known wavelengths of the irradiated light may then be analyzed to provide medical information representative of the physiological region of interest. However, spectroscopic devices may not able to evaluate precise regions of interest, such as individual blood vessels.

### BRIEF DESCRIPTION OF THE DRAWINGS

Advantages of this disclosure may become apparent upon reading the following detailed description and upon reference to the drawings in which:

FIG. 1 is a block diagram of a patient monitor and spectroscopic sensor in accordance with an embodiment;

FIG. 2 depicts an ultrasound modulated optical measurement in accordance with an embodiment;

FIG. 3 depicts a focused ultrasound modulated optical measurement in accordance with an embodiment; and

FIG. 4 depicts a flowchart of a method for observing localized regions of interest.

### DETAILED DESCRIPTION

One or more specific embodiments of the present disclosure will be described below. In an effort to provide a concise description of these embodiments, not all features of an actual implementation are described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

In certain medical contexts it may be desirable to ascertain various localized physiological parameters, such as parameters related to individual blood vessels or other discrete components of the vascular system. Examples of such parameters may include oxygen saturation, hemoglobin concentration, perfusion, and so forth, for an individual blood vessel. One approach to measuring such localized parameters is referred to as ultrasound-modulated optical (UO) spectroscopy.

UO spectroscopy utilizes a combination of both sound waves and light waves directed into a patient's tissue. The sound waves create an ultrasonic focal volume at a specific region of interest. The interaction of the light waves with the ultrasonic energy causes a change in the spatial distribution of the particles as well as a change in the refractive index of the scattering particles. Accordingly, the light waves passing through the ultrasonic focal volume are modulated by a combination of ultrasound-induced particle displacement and changes in the refractive index of the particles. Certain localized measurements may then be computed by filtering out unmodulated light and using only the ultrasound-modulated light. In particular, the light energy directed into the tissue may be provided at particular wavelengths that correspond to the absorption profile of one or more blood or tissue constituents of interest.

One problem that may arise in UO spectroscopy may be attributed to the tendency of the emitted light to diffuse or scatter in the tissue of the patient. As a result, light emitted toward an internal structure or region, such as a blood vessel, may be diffused prior to reaching the intended focal region so that amount of light reaching the ultrasound's focal region and modulated by the ultrasound field is less than desired. Therefore, due to the diffusion of the light, less light may be available to be absorbed by the constituent of interest in the focal region, thus reducing the observed light generated at the region of interest, such as a blood vessel. In certain embodiments of the present disclosure, the emitted light may be focused on an internal region of interest by spatially modulating the illuminating light before it enters the tissue so as to reduce or eliminate the effects of light diffusion. Accordingly, a spatially-modulated UO spectroscopy system may be capable of more precise measurements of a variety of vessel-specific physiological parameters, which may be desired for many applications.

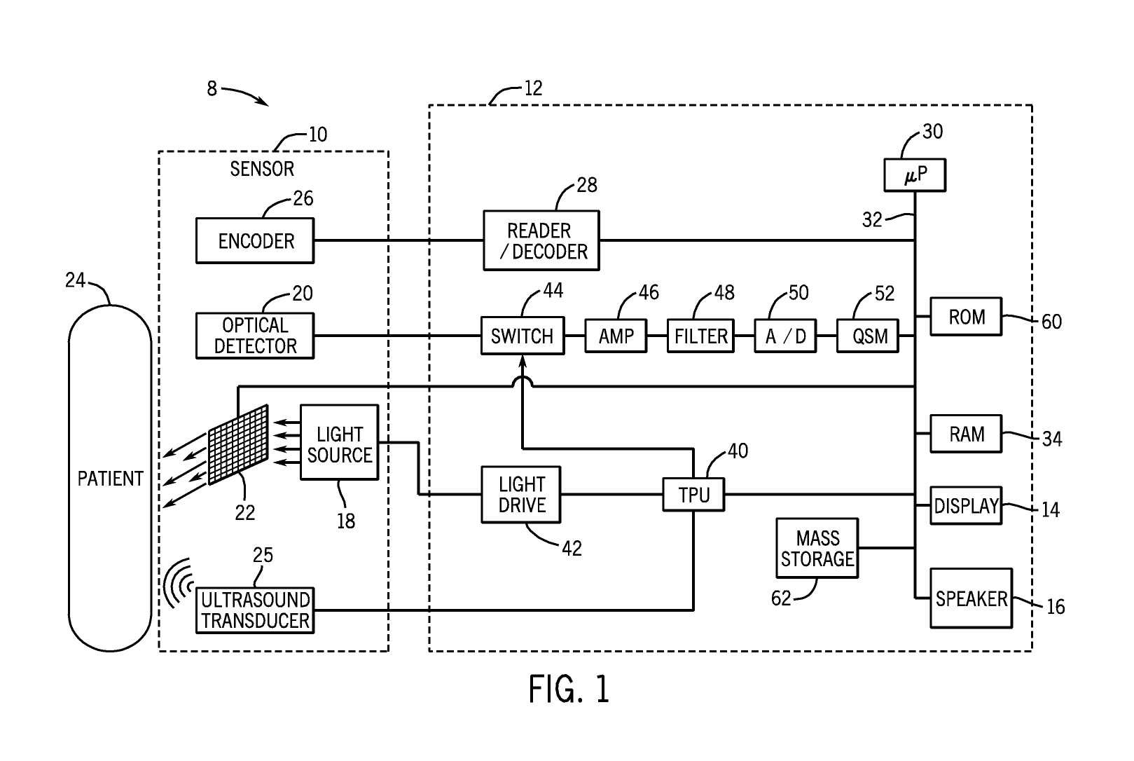

With this in mind, FIG. 1 depicts a block diagram of a UO spectroscopy system 8 in accordance with embodiments of the present disclosure. The system 8 includes a UO spectroscopy sensor 10 and a monitor 12. The sensor 10 may emit spatially modulated light at certain wavelengths into a patient's tissue. The sensor 10 may also emit sound waves (e.g., ultrasound waves) at certain frequencies, creating an ultrasound focal spot. The monitor 12 may be capable of calculating physiological characteristics based on light signals received from the sensor 10 that correspond to the observed ultrasound-modulated light waves. The monitor 12 may include a display 14 and/or a speaker 16 which may be used to convey information about the calculated physiological characteristics to a user. The sensor 10 may be communicatively coupled to the monitor 12 via a cable or, in some embodiments, via a wireless communication link.

In one example, the sensor 10 may include a light source 18 and an optical detector 20, such as a charge-coupled device (CCD) or an interferometer. The present discussion generally describes the use of continuous wave light sources to facilitate explanation. However, it should be appreciated that the spectrophotometric sensor 10 may also be adapted for use with other types of light sources, such as pulsed light sources, in other embodiments. In certain embodiments, the light source 18 may be associated with one or more optical fibers for conveying light from one or more light generating components to the tissue site.

The UO spectroscopy sensor 10 may include a light source 18 and an optical detector 20 that may be of any suitable type. For example, in certain embodiments, the light source 18 may include a laser diode or a vertical cavity surface emitting laser (VCSEL). The laser diode may be a tunable laser, such that a single diode may be tuned to various wavelengths corresponding to a number of different absorbers of interest in the tissue and blood. That is, the light may be any suitable wavelength or wavelengths (such as a wavelength between about 500 nm to about 1100 nm or between about 600 nm to about 900 nm) that is absorbed by a constituent of interest in the blood or tissue. For example, wavelengths between about 500 nm to about 600 nm, corresponding with green visible light, may be absorbed by deoxyhemoglobin and oxyhemoglobin. In other embodiments, red wavelengths (e.g., about 600 nm to about 700 nm) and infrared or near infrared wavelengths (e.g., about 800 nm to about 1100 nm) may be used. In one embodiment, the selected wavelengths of light may penetrate between 1 mm to 15 cm into the tissue of the patient 24.

An ultrasound transducer 25 may be employed to direct ultrasound waves into the patient tissue 24. The ultrasonic waves may be emitted by the ultrasound transducer 25 at any suitable frequency, such as from 0.5 MHz to 20 MHz or more. The ultrasonic frequency may be chosen based on a desired imaging depth. For example, lower frequencies may increase the imaging depth, while higher frequencies may decrease the imaging depth. In one embodiment, the ultrasound transducer 25 may emit the ultrasonic waves in pulses lasting between approximately 1 ns and 1 sec. The ultrasound energy emitted into the patient tissue 24 may result in a peak pressure of between approximately 0.5 MPa and 5 MPa at a focal area of between approximately 1 mm and 15 mm. In such an embodiment, the limited duration and power of the ultrasound waves may prevent excessive energy from being directed into the patient tissue 24 while still emitting ultrasound waves of sufficient energy into the region of interest to generate the desired acoustic-optical modulation. The ultrasonic waves may be timed so as to coincide with light waves emitted from the light source 18.

The light emitted by the light source 18 may be spatially modulated, such as via a modulator 22. For example, in one embodiment, the modulator 22 may be a spatial light modulator, such as a Holoeye® LC-R 2500 liquid crystal spatial light modulator. In one such embodiment, the spatial light modulator may have a resolution of 1024×768 pixels or any other suitable pixel resolution. During operation, the pixels of the modulator 22 may be divided into subgroups (such as square or rectangular subarrays or groupings of pixels) and the pixels within a subgroup may generally operate together. For example, the pixels of a modulator 22 may be generally divided into square arrays of 10×10, 20×20, 40×40, or 50×50 pixels. In one embodiment, each subgroup of pixels of the modulator 22 may be operated independently of the other subgroups. The pixels within a subgroup may be operated jointly (i.e., are on or off at the same time) though the subgroups themselves may be operated independently of one another. In this manner, each subgroup of pixels of the modulator 22 may be operated so as to introduce phase differences at different spatial locations within the emitted light. That is, the modulated light that has passed through one subgroup of pixels may be at one phase and that phase may be the same or different than the modulated light that has passed through other subgroups of pixels, i.e., some segments or portions of the modulated light wavefront may be ahead of or behind other portions of the wavefront. In one embodiment, the modulator 22 may be associated with additional optical components (e.g., lenses, reflectors, refraction gradients, polarizers, and so forth) through which the spatially modulated light passes before reaching the tissue of the patient 24.

In another example, the optical detector 20 may be an interferometry device. In these examples, a modulated portion of the detected light may be detected via the interferometry device (e.g., Fabry-Perot interferometer) which filters through the modulated light, and generates an electrical signal that may be processed by the monitor 12. The use of the Fabry-Perot interferometer may enable a spectral filtering of the observed light useful in separating the modulated light from the unmodulated light. Additionally, the optical detector 20 may include spectrometric techniques capable of measuring the intensity of light at a variety of specific light waves, such as light waves between 500 nm and 1000 nm.

In another example, the optical detector 20 may be an interferometry device. In these examples, a change in phase of the detected light may be detected via the interferometry device (i.e., interferometer) which generates an electrical signal that may be processed by the monitor 12. The interferometer may use a thin film and an optical fiber. The use of the thin film as the optical detecting surface allows high sensitivity to be achieved, even for films of micrometer or tens of micrometers in thickness. In one embodiment, the thin film may be a 0.25 mm diameter disk of 50 micrometer thickness polyethylene terepthalate with an at least partially optically reflective (e.g., 40% reflective) aluminum coating on one side and a mirror reflective coating on the other (e.g., 100% reflective) that form the mirrors of the interferometer. The optical fiber may be any suitable fiber, such as a 50 micrometer core silica multimode fiber of numerical aperture 0.1 and an outer diameter of 0.25 mm. Additionally, the optical detector 20 may include spectrometric techniques capable of measuring the intensity of light at a variety of specific wavelengths, such as wavelengths between 500 nm and 1000 nm.

The UO sensor 10 may include a memory or other data encoding component, depicted in FIG. 1 as an encoder 26. For example, the encoder 26 may be a solid state memory, a resistor, or combination of resistors and/or memory components that may be read or decoded by the monitor 12, such as via reader/decoder 28, to provide the monitor 12 with information about the attached sensor 10. For example, the encoder 26 may encode information about the sensor 10 or its components (such as information about the light source 18, the optical detector 20, and/or the ultrasound transducer 25). Such encoded information may include information about the configuration and/or location of the UO sensor 10; information about the type of lights source(s) 18 present on the sensor 10; information about the wavelengths, frequencies, and energies of the ultrasonic and light waves that the light source(s) 18 and the ultrasound transducer 25 are capable of emitting; information about the properties of the optical detector 20; and so forth. The stored information may also include algorithms, as described in more detail below, suitable for manipulating the ultrasonic waves and/or the emitted light radiated into the tissue.

In one implementation, signals from the optical detector 20 (and decoded data from the encoder 26, if present) may be transmitted to the monitor 12. The monitor 12 may include data processing circuitry (such as one or more processors 30, application specific integrated circuits (ASICS), or so forth) coupled to an internal bus 32. Also connected to the bus 32 may be a RAM memory 34, a speaker 16 and/or a display 14. In one embodiment, a time processing unit (TPU) 40 may provide timing control signals to light drive circuitry 42, which controls operation of the light source 18 and the ultrasound transducer 25, such as to control when, for how long, and/or how frequently the light source 18 and/or the ultrasound transducer 25 is activated, and if multiple light sources are used, the multiplexed timing for the different light sources.

The TPU 40 may also control or contribute to operation of the optical detector 20 such that timing information for data acquired using the optical detector 20 may be obtained. Such timing information may be used in interpreting the emitted light wave data and/or in generating physiological information of interest from such optical data. For example, the timing of the optical data acquired using the optical detector 20 may first be associated with the light emission profile of the light source 18 during ultrasound emission. The TPU 40 may then instruct the ultrasound transducer 25 to stop ultrasound emission. The optical data may then be acquired based on the light emission profile light source 18 without any ultrasound manipulation. Likewise, in one embodiment, data acquisition by the optical detector 20 may be gated, such as via a switching circuit 44, to account for differing aspects of light emission. For example, operation of the switching circuit 44 may allow for separate or discrete acquisition of data that corresponds to different respective wavelengths of light emitted at different times.

The received signal from the optical detector 20 may be amplified (such as via amplifier 46), may be filtered (such as via filter 48), and/or may be digitized if initially analog (such as via an analog-to-digital converter 50). The digital data may be provided directly to the processor 30, may be stored in the RAM 34, and/or may be stored in a queued serial module (QSM) 52 prior to being downloaded to RAM 34 as QSM 52 fills up. In one embodiment, there may be separate, parallel paths for separate amplifiers, filters, and/or A/D converters provided for different respective light wavelengths or spectra used to generate the optical data.

The data processing circuitry (such as processor 30) may derive one or more physiological characteristics based on data generated by the spectrophotometric sensor 10. For example, based at least in part upon data received from the optical detector 20, the processor 30 may calculate the amount or concentration of a constituent of interest in a localized region of tissue or blood using various algorithms. In certain embodiments, these algorithms may include speckle processing algorithms, such as speck contrast algorithms, suitable for comparing a first ultrasound-modulated image with a second non-modulated image so as to derive a third, two-dimensional (2D) image. The derived 2D image may be suitable for deriving a variety of localized observations such as microcirculatory features, including arterioles and venules. Further, 2D and 3D images may be created by analyzing the optical signals. Such analysis may involve techniques that can incorporate the observation that different types of constituents absorb light at different wavelengths. In addition, in one embodiment the data processing circuitry (such as processor 30) may communicate with the TPU 40 and/or the light drive 42 to spatially modulate the wave front of light emitted by the light source 18 based on one or more algorithms, as discussed herein.

In one embodiment, processor 30 may access and execute coded instructions, such as for implementing the algorithms discussed herein, from one or more storage components of the monitor 12, such as the RAM 34, a ROM 60, and/or the mass storage 62. Additionally, the RAM 34, ROM 60, and/or the mass storage 62 may serve as data repositories for information such as modulation ratios, coefficient curves, and so forth. For example, code encoding executable algorithms may be stored in the ROM 60 or mass storage device 62 (such as a magnetic or solid state hard drive or memory or an optical disk or memory) and accessed and operated according to processor 30 instructions using stored data. Such algorithms, when executed and provided with data from the sensor 10, may calculate a physiological characteristic as discussed herein (such as the type, concentration, and/or amount of a constituent of interest). Once calculated, the physiological characteristic may be displayed on the display 14 for a caregiver to monitor or review.

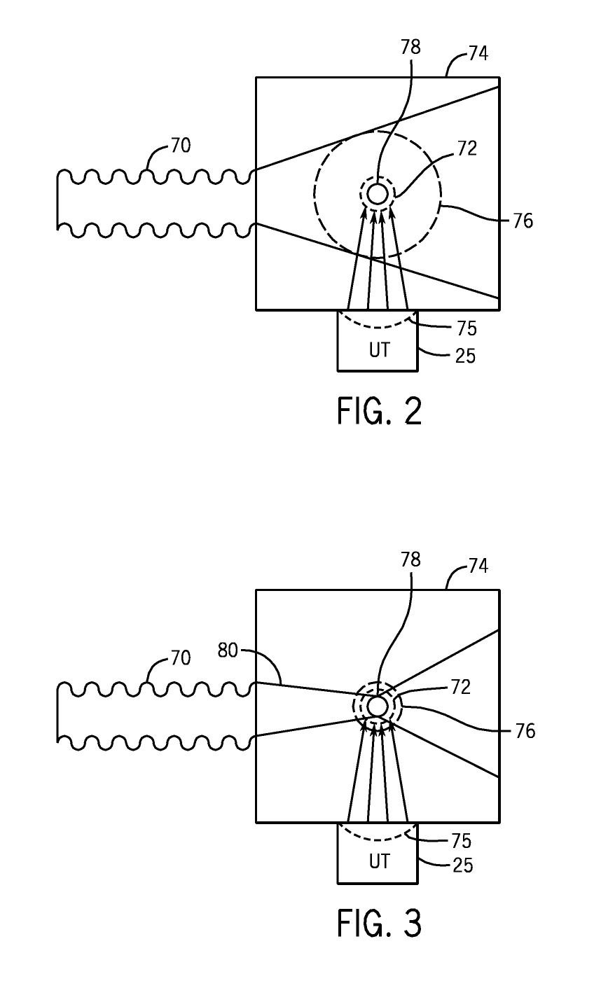

With the foregoing system discussion in mind, light emitted by the light source 18 of the spectrophotometric sensor 10 may be used to generate optical signals in proportion the amount of an absorber (e.g., a constituent of interest) in a targeted localized region. However, as noted above, the emitted light may be scattered upon entering the tissue, with the amount of scatter or dispersion increasing as the light penetrates deeper into the tissue. Thus, for localized regions or structures of interest, such as blood vessels, the greater the depth of such vessels beneath the tissue surface, the greater the dispersion of the emitted light before reaching the localized region or structure. Further, wider separation between the light emitter and the light detector may increase the depth of light penetration and corresponding detection. For example, referring to FIG. 2, a light 70 and an ultrasound focal area 72 may be generated by the ultrasound transducer 25 and directed into a tissue 74. The ultrasound focal area 72 may be created by focusing ultrasound waves, for example, though the use of a spherical dish 75 included in the ultrasound transducer 25. As the light 70 diffuses in the tissue 74, a portion of the diffused light may intersect the ultrasound focal area 72. An intersecting portion 76 of the light 70 may be modulated by ultrasound waves, for example, due to changes in the refractive index of the particles in the tissue, or due to a change in the spatial distribution of the particles. The modulated photons 76 may then carry certain optical properties, such as optical absorption and scattering coefficients, of a localized region of interest 78.

Certain speckle processing techniques, such as a speckle contrast detection, may be applied to the observed light as described in more detail below with respect to FIG. 3, to determine the measurements of interest. However, the light 70 may begin to disperse upon entering the tissue 74. As a result, the intensity and/or fluence of the emitted light incident upon the localized region of interest 78, such as blood vessel, may be reduced, resulting in less absorption by the constituent of interest within the localized region 78 with proportionately fewer modulated photons 76 being generated. This may yield a relatively low signal strength at the optical detector 20 relative to the noise (i.e., low signal-to-noise ratio) associated with the measurement.

Turning to FIG. 3, in one embodiment, the strength of the measured signal may be increased by focusing the light 70 on the region of interest 78, as denoted by focused beam 80. Such focusing may result in less dispersal or scattering of the light prior to reaching the region of interest 78 and correspondingly greater intensity and/or fluence of the light at the region of interest 78. The ultrasound focal area 72 generated by the ultrasound transducer 25 may thus intersect the more focused beam 80. As a result, more absorption of light by the constituent of interest may occur in the region of interest 78, yielding more modulated photons 76 with a corresponding higher signal-to-noise ratio at the optical detector 20.

The light 70 may first be spatially modulated using a liquid crystal phase modulator or other suitable modulator 22. That is, the light 70 may be focused on one or more concurrent focal points by spatially modulating the light 70 to yield an inverse wave diffusion effect upon entering the scattering medium, i.e., the patient tissue. In effect, multi-path interference may be employed so that the scattering process itself focuses the emitted light onto the desired focal point or points. In particular, to the extent that at any given time the disorder in a medium is fixed or determinable, light scattering in the medium is deterministic and this knowledge may be utilized to modulate the emitted light such that the resulting scatter in the medium results in the light being concentrated or focused on a desired region of interest.

For example, to the extent that a light wave may have a planar wavefront, a spatially modulated light wave, as discussed herein, may have a wavefront that is not planar and instead may be shaped by breaking the wavefront up into numerous sub-planes (e.g., square or rectangular segments) that are not all at the same phase, such that different portions of the wavefront reach the tissue surface at different times. The operation of the modulator 22 may be updated or iterated based upon feedback from the optical detector 20. For example, in one embodiment the signals generated by the optical detector 20 may be processed by a processor 30 which may in turn evaluate the processed signal in accordance with one or more algorithms or thresholds (such as a signal-to-noise threshold) and adjust operation of the modulator 22 accordingly. In one embodiment adaptive learning algorithms or other suitable analysis algorithms (e.g., neural networks, genetic algorithms, and so forth) may be employed to evaluate the processed signal and to make adjustments to the modulation.

In one example, an algorithm may be stored in the memory 34 and executed by the processor 30 to generate the inverse diffusion wavefront. One such algorithm may utilize the linearity of the scattering process in the tissue to generate the diffusion wavefront. For example, in one embodiment, the inverse diffusion wavefront may be generated in accordance with the equation:

$${E}_{m} = {\sum }_{n = 1}^{N} {t}_{mn} {A}_{n} {e}^{{iφ}_{n}} \qquad ( 1 )$$

where Eₘ is the linear combination of the fields coming from N different wavefront segments generated by the modulator 22, Aₙ is the amplitude of the light reflected from segment n, φₙ is the phase of the light reflected from segment n, and tₘₙ is the scattering in the sample and propagation through the optical system. In accordance with such an equation, the magnitude of Eₘ may be maximized when all terms are in phase. The optimal phase for a segment, n, of the light wavefront at a given time may be determined by cycling its phase from 0 to 2π while the phase of other segments is held constant. This process may then be repeated for each segment. The optimal phase for each segment for which the target intensity is highest may then be stored. Once the optimized phase is known for each segment of the wavefront, the modulator 22 may be programmed based on the stored values such that differential activation of the pixels or subgroups of pixels defined for the modulator 22 (such as for a liquid crystal phase modulator) spatially modulates the light incident upon the modulator 22. That is, differential adjustment of the opacity of elements defined by the modulator 22 (such as square or rectangular groupings of pixels of a liquid crystal element) may yield a light with a wavefront in which different segments or portions of the wavefront are out of phase, i.e., staggered with respect to one another. When the resulting spatially modulated light is transmitted through the tissue, the contributions attributable to each modulated portion of the wavefront of the light may constructively interfere with one another to yield the desired light intensity at the localized region of interest, as depicted in FIG. 3.

While the preceding describes one implementation for generating a spatially modulated wavefront, such a wavefront may also be generated by an algorithm stored in the memory 34 and executed by the processor 30 that models the optical field E at a point r_{b} within a medium in accordance with:

E(r_{b})=∫g(r_{b},rₐ)φ(rₐ)d³rₐ (2)

in which g is Green's function describing propagation from rₐ to point r_{b}. In an embodiment, each segment of the phase modulator is treated as a planar source having amplitude A and phase φ. If the phase modulator is assumed to be illuminated uniformly, the amplitudes A at each segment may be assumed to be equal. By integrating the surface area S of each of the N segments, Equation (2) may be represented as:

$$E ( {r}_{b} ) = {\sum }_{a}^{N} {\int }_{{S}_{a}} g ( {r}_{b} , {r}_{a} ) {d}^{2} {r}_{a} A {e}^{iφ a} \qquad ( 3 )$$

which in turn yields

$$E ( {r}_{b} ) = A {\sum }_{a}^{N} {g}_{ba} {e}^{iφ a} . \qquad ( 4 )$$

Changing the phase of a segment a of the phase modulator 22 while holding the phase of other segments unchanged causes the intensity I at point r_{b} to respond in accordance with:

I(r_{b})≡|E(r_{b})|²=I_{0b}+2ARe(E*_{\tilde{b}ã}g_{ba}e^{iφa}) (5)

in which:

I_{0b}≡|E_{\tilde{b}ã}|²+A²|g_{ba}|² (6)

and

$${E}_{b \overline{a}} ≡ A {\sum }_{{a}^{′} \neq a}^{N} {g}_{{ba}^{′}} {e}^{{iφ}_{a}^{′}} \approx E ( {r}_{b} ) . \qquad ( 7 )$$

Where the number of segments N is large E_{\tilde{b}ã}≈E(r_{b}) and is therefore essentially the same across all segments. By analyzing each segment a in this manner, the coefficients g_{ba} may be measured up to an unknown common prefactor E(r_{b}). By determining the coefficients g_{ba}, the optical field at point r_{b} (e.g., E(r_{b})) may be maximized by setting φₐ equal to −arg(g_{ba}) for each of the segments. This combination of segment phases thus can yield an aggregate light intensity maximum at the region of interest:

$${E}_{max} ( {r}_{b} ) = A {\sum }_{a}^{N} \vert {g}_{ba} \vert \qquad ( 8 )$$

in which the different light channels associated with each channel will undergo constructive interference to reach the region of interest.

The amount of intensity enhancement observed at the localized region 78 may be related to the numbers of segments or regions into which the wavefront of the light 70 is broken. To the extent that the constants tₘₙ are statistically independent and obey a circular Gaussian distribution, the expected enhancement, κ, may be represented as:

$$η = \frac{π}{4} ( N - 1 ) + 1 \qquad ( 9 )$$

where κ is the ratio between the enhanced light intensity at the region of interest and the average light intensity at the region of interest prior to enhancement.

In one example, a speckle-contrast detection scheme may be used to measure the modulated light component by measuring changes in speckle statistics between ultrasound-on and ultrasound-off states, In this example, the optical sensor 20 may capture both ultrasound modulated and unmodulated light. Additionally, the optical sensor 20, such as a CCD, may be time gated to capture modulated light before an acoustic radiation force

(ARF) caused by the ultrasound emission has an opportunity to accumulate significant tissue displacement. Such time gating may reduce the effects of shear-wave image degradation, and thus, may enable a higher signal to noise ratio, Accordingly, a delayed trigger may be used to set the time generation of the ultrasound pulses. Further, the emission of the ultrasound pulse may be set to so as to coincide with the arrival of the emitted light at a given depth z in the patient tissue. That is, a time delay t may be set such that the ultrasound pulse propagates to a desired depth in the region of interest at the same time that the next optical pulse arrives in the region of interest, Indeed, such a delay t may be suitable for controlling the depth z of the ultrasonic focal point.

Speckle arises when coherent light scattered over a rough surface or tissue is detected by an intensity detector that has a finite aperture. Speckle contrast is related to temporal field auto correlation functions, which in turn are related to the acoustic modulation properties of the acoustically modulated light. A first acoustically modulated image may be compared to a second unmodulated image, for example, by using the following speckle contrast equation:

$$C = \frac{σ}{{〈 \overline{{I}_{s}} 〉}_{p}^{′}} \qquad ( 10 )$$

Where σ is the standard deviation of the optical speckle pattern, averaged over all the CCD pixels p and [symbol] I [symbol] ₚ is the mean speckle intensity over all pixels p. The use of equation (10) may allow for a comparison of the acoustically modulated image from the unmodulated image by comparing the speckles contrasts between the two images. That is, a first speckle contrast image representing the unmodulated image may be subtracted from a second speckle contrast image representing the modulate image, resulting in a third image that has an improved image quality in a localized region of interest. Indeed, optical features in the sub-millimeter range may be detected. Further, a set of 2D images at different tissue depths may be created, for example, by varying the delay t, A 3D image may be constructed by layering the set of 2D images, each layer corresponding to a different tissue depth.

In another example, a comparison between optical signals received from two or more wavelengths may be used to quantitatively derive certain measurements of interest such as hemoglobin, oxygen saturation, blood perfusion, and so forth. In this example, the region of interest may first be irradiated with the light 70 at an optical wavelengths λ₁ corresponding to a constituent of interest C₁ followed by a second irradiation of the light 70 at an optical wavelength λ₂ corresponding to a second constituent of interest C₂. The two acoustically modulated optical signals corresponding to the light waves λ₁ and λ₂ may then be acquired and processed. The signals may first be normalized by using an average signal corresponding to, for example, a theoretical tissue devoid of embedded objects. Absorption coefficients for the constituents of interest may then be used as follows:

$$\frac{[ {C}_{1} ]}{[ {C}_{1} ] + [ {C}_{2} ]} = \frac{{μ}_{a} ( {λ}_{2} ) {ɛ}_{{C}_{2}} ( {λ}_{1} ) - {μ}_{a} ( {λ}_{1} ) {ɛ}_{{C}_{2}} ( {λ}_{2} )}{{μ}_{a} ( {λ}_{1} ) Δɛ ( {λ}_{2} ) - {μ}_{a} ( {λ}_{2} ) Δɛ ( {λ}_{1} )} \qquad ( 11 )$$

where [C₁] is the approximate relative concentration of the first constituent of interest (e.g., deoxyhemoglobin) in a carrier medium such as blood, [C₂] is the approximate relative concentration of the second constituent of interest (e.g., oxyhemoglobin) in the carrier medium, ε_{C}_{₁}is the absorption coefficient of the first constituent of interest, ε_{C}_{₂} is the absorption coefficient of the second constituent of interest, μₐ is the measured relative absorption between the first and the second constituents, and Δε=ε_{C}_{₁}−ε_{C}₂. For example, the ratio of deoxyhemoglobin to oxyhemoglobin may be found by utilizing their known or measured absorption coefficients in equation (11). By varying the wavelengths used for observation, various different types of constituent measurements may be derived based on the observation that different types of constituents absorb light at different wavelengths.

Thus, in accordance with the present disclosure, emitted light may be spatially and acoustically modulated so as to converge on a region of interest within an otherwise scattering medium (e.g., tissue). In the context of ultrasound-modulated optical spectroscopy, such convergence may be used to increase the fluence of light at the internal region of interest (e.g., light absorber) and to, thereby, improve the signal-to-noise ratio of the ultrasound-modulated optical signals. That is, focusing the emitted light on the internal region (such as by spatial modulation of the respective light wavefronts) modulated by an ultrasound field generates more modulated optical signal, thereby improving the measurement signal-to-noise ratio. Such techniques allow for precise measurements in individual vasocirculatory structures. For example, hemoglobin concentration and oxygen saturation (i.e., percentage of oxygen in the blood) measurements may now be derived in localized regions of interest. The optical absorption spectra of oxygenated hemoglobin (HbO₂) and deoxygenated hemoglobin (Hb) may be used to determine precise quantities of these two chromophores in the area being observed by irradiating the area with light near certain wavelengths such as 660 nm and 900 nm. The chromophores preferentially absorb light at certain wavelengths resulting in enhanced or reduced ultrasonic responses based on which wavelength is currently used to irradiate the tissue. The resulting optical responses may be analyzed to measure hemoglobin concentration as well as oxygen saturation in arterial and venous conduits. Such measurements allow for determination of conditions such as anemia, iron deficiency, low or high blood oxygenation, and so forth.

Imaging modalities may also be employed that allow for enhanced detail and image resolution of the tissue site under observation. Indeed, detailed in vivo 2D and 3D imaging may be created by deriving an image based on the type, amount concentration, and/or the location of the various tissue constituents observed by the UO spectroscopy system 8. The signals resulting from such observations may be processed by the techniques disclosed above, such as the algorithmic techniques, to derive an image corresponding to the image of the area under observation. Such imaging may be useful for capillary mapping, skin melanoma detection, and so forth. It is thus possible to observe the micro-circulation of blood among individual arterioles and venules, thus enabling the characterization of blood flow and tissue perfusion (e.g., hydrostatic pressure measurements, osmotic pressure measurements) at a capillary level. Additionally, soft brain tissues having different optical absorption properties may be observed by the techniques disclosed herein. For example, an optical response between a lesion area and a healthy area may be significantly different. Accordingly, a lesion area may be identified and imaged during in vivo examination of brain tissue using the UO spectroscopy system 8.

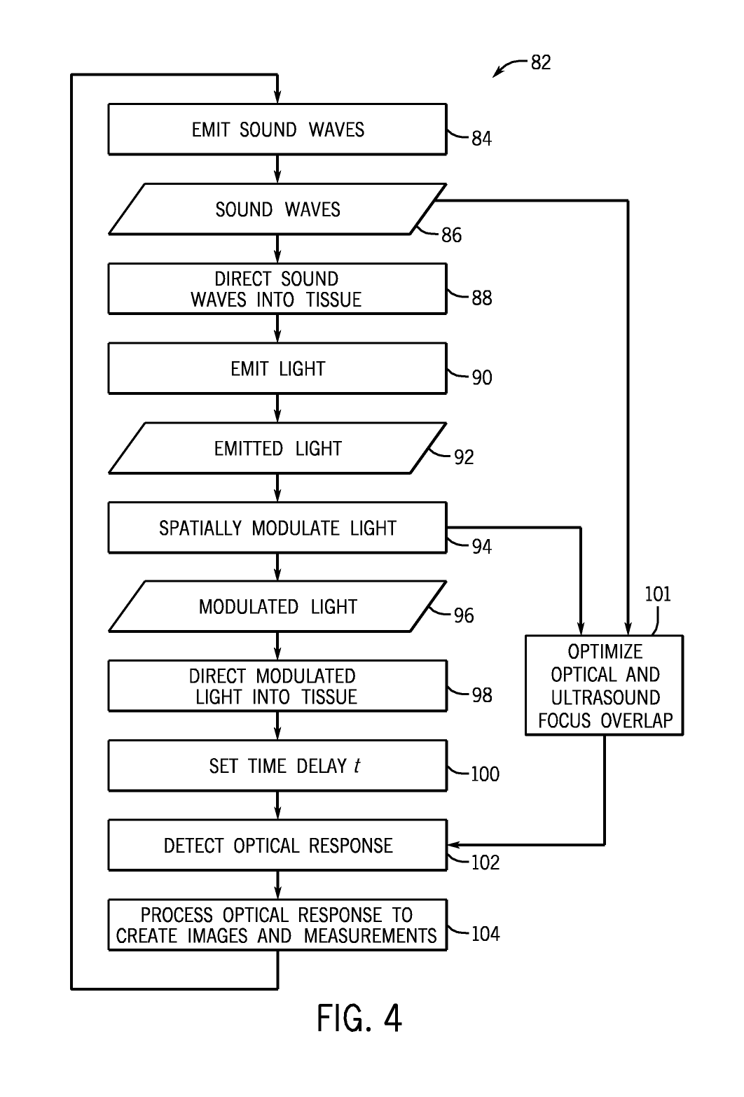

Turning to FIG. 4, the figure is a flowchart of a logic 82 that may be employed to measure very precise regions of interest in a tissue. In certain embodiments, an ultrasound transducer may be driven so as to emit sounds waves (block 84). The emitted sound waves 86 may then be directed into the patient's tissue (block 88). A light, such as a light at specific wavelengths may also be emitted (block 90) by a light emitter such as a tunable laser. The emitted light 92 may then be redirected into a spatial light modulator for further focusing through spatial modulation (block 94). The spatial light modulator is capable of altering the wavefront of the incident light as described above in relation to FIG. 3 such that the wavefront may be broken up into numerous sub-planes that are not all at the same phase. The spatially modulated light 96 may then be emitted onto the tissue sample (block 98).

The light incident upon the tissue sample may encounter the sound waves and experience further acoustic modulation. More specifically, the light waves passing through the ultrasonic focal volume are phase modulated by a combination of ultrasound-induced particle displacement and changes in the refractive index of the particles. In certain embodiments, a time delay t may be set (block 100) to enable the creation of an ultrasonic pulse within the focal depth at a given depth z. In these embodiments, the sound waves 86 coincide with the arrival of a spatially modulated light 98 at depth z of the patient tissue at approximately time t. In certain embodiments, the overlap between the optical and ultrasound focus may be optimized (block 101). That is, the optical and/or ultrasound beams may be adjusted so as to maximize the overlap between the optical and ultrasound focal areas. In these embodiments, various adaptive algorithms may be used that take into account previously detected signal data (e.g., optical signal data) to vary the optical and/or ultrasound focal areas so as to improve the optical and ultrasound focus overlap. Accordingly, low overlap data may be corrected, resulting in subsequent improvements in the detected light.

The resultant light at time t can be detected, for example, by the optical detector 20 (block 102). The optical detector 20 is capable of converting the detected light waves into electric signals. In certain embodiments, the electrical signals are processed by a variety of algorithms as described above so as to determine a concentration or quantity measure of light absorbers within a localized region of the tissue (block 104). As mentioned previously, the algorithms are capable of using a variety of spatial modulation enhancement techniques to observe the localized region. Similarly, techniques such as speckle processing algorithms may be employed to process the acoustically modulated components of the signal. The processed signal may be used to determine localized measurements of certain physiologic parameters such as hemoglobin concentration and oxygen saturation. Other measurements may be obtained based on microcirculatory observations, such as hydrostatic pressure measurements and osmotic pressure measurements. Further, imaging modalities may be employed to produce in vivo images such as capillary maps, tissue maps, brain lesion images, and so forth, based on, for example, differing absorption contrasts among tissue regions. Indeed, the techniques disclosed herein allow for very precise imaging of tissue as well as for obtaining spectrophotometric measurements of highly localized regions of interest. The logic 82 may then iteratively emit light and sound waves, modulate the emitted light, and process the resulting signal so as to continuously observe the region of interest, as illustrated.

While the disclosure may be susceptible to various modifications and alternative forms, specific embodiments have been shown by way of example in the drawings and have been described in detail herein. However, it should be understood that the embodiments provided herein are not intended to be limited to the particular forms disclosed. Indeed, the disclosed embodiments may be applied to various types of medical devices and monitors, as well as to electronic device in general. Rather, the various embodiments may cover all modifications, equivalents, and alternatives falling within the spirit and scope of the disclosure as defined by the following appended claims.

What is claimed is:

1. An ultrasound-modulated optical system, comprising:
a light emitting component capable of emitting one or more wavelengths of light;
a first light modulating component capable of spatially modulating the light emitted by the light emitting component;
a second light modulating component capable of acoustically modulating the light emitted by the light emitting component or by the first light modulating component;
an optical detector capable of detecting light; and
a processor capable of deriving a physiologic measurement or of creating an image based on the light detected by the optical detector.

2. The ultrasound-modulated optical system of claim 1, wherein the first light modulating component comprises a liquid crystal spatial light modulator.

3. The ultrasound-modulated optical system of claim 1, wherein the second light modulating component comprises an ultrasound transducer configured to emit ultrasound waves.

4. The ultrasound-modulated optical system of claim 3, wherein the processor is configured to process observations of the acoustically modulated light after a time delay, and wherein the time delay is correlative to an observation depth.

5. The ultrasound-modulated optical system of claim 1, wherein the light emitting component comprises one or more light emitting diodes, one or more laser diodes, a vertical cavity surface emitting laser, or any combination thereof.

6. The ultrasound-modulated optical system of claim 1, wherein the processor derives the physiologic measurement or creates the image by using a speckle processing algorithm.

7. The ultrasound-modulated optical system of claim 1, wherein the light emitting component, the first light modulating component, the second light modulating component, the optical detector, or any combination thereof, are provided in a sensor body.

8. The ultrasound-modulated optical system of claim 1, comprising a monitor communicatively coupled with the light emitting component, the first light modulating component, the second light modulating component, and the optical detector, wherein the processor is provided in the monitor, and wherein the monitor is capable of controlling the light emitting component, the first light modulating component, the second light modulating component, and the optical detector.

9. The ultrasound-modulated optical system of claim 4, wherein the physiologic measurement comprises a hemoglobin concentration or an oxygen saturation.

10. The ultrasound-modulated optical system of claim 1, wherein the optical detector comprises a charge-coupled device (CCD), or an interferometer, or a combination thereof.

11. A ultrasound-modulated optical sensor, comprising:
a light source capable of emitting light at one or more wavelengths;
a first modulator capable of spatially modulating wavefronts associated with the emitted light, such that each wavefront exhibits different phases at different locations;
a second modulator capable of acoustically modulating the light emitted by the light source or by the first modulator; and
an optical detector capable of generating a signal in response to the modulated light wherein a physiologic measurement or an image may be derived based on the signal generated by the optical detector.

12. The ultrasound-modulated optical sensor of claim 11, wherein the one or more respective wavelengths are between approximately 500 nm to approximately 1,100 nm.

13. The ultrasound-modulated optical sensor of claim 11, wherein the first modulator comprises a plurality of subgroups of pixels,

14. The ultrasound-modulated optical sensor of claim 11, wherein the second modulator comprises an ultrasound transducer.

15. A ultrasound-modulated optical monitor, comprising:
a processor;
a first demodulator capable of demodulating a spatially modulated light; and
a second demodulator capable of demodulating an acoustically modulated light, wherein the processor is configured to derive a physiologic measurement or to create an image based on a signal generated by an optical detector and demodulated by the first and second demodulators.

16. The monitor of claim 15, wherein the spatially modulated light comprises spatially modulating wavefronts such that each wavefront exhibits different phases at different locations.

17. The monitor of claim 15, wherein the acoustically modulated light comprises light modulated by a change in the spatial distribution of tissue particles, light modulated by a change in the refractive index of the tissue particles, or a combination thereof.

18. A method for processing optical signals, comprising:
generating light at one or more wavelengths;
spatially modulating the generated light;
generating ultrasound waves;
emitting the spatially modulated light and the ultrasound waves towards a medium to generate ultrasonically modulated light in the medium;
detecting the ultrasonically modulated light within the medium;
generating a signal corresponding to the detected ultrasonically modulated light; and
processing the signal to generate one or more measurements related to the presence of a light absorber within the medium.

19. The method of claim 18, wherein the detecting the ultrasonically modulated light within the medium comprises setting a time delay, and wherein the time delay is correlative to an observation depth.

20. The method of claim 18, comprising processing the signal to generate one or more images related to the presence of the light absorber within the medium.
